(19) United States
(12) Patent Application Publication
(10) Pub. No.: US 2026/0133207 A1
(43) Pub. Date: May 14, 2026

(54) MOLECULAR MARKER FOR DIAGNOSIS, PROGNOSIS EVALUATION, AND TREATMENT OF CARDIAC FIBROSIS

(71) Applicants: FUWAI CENTRAL CHINA CARDIOVASCULAR HOSPITAL, Zhengzhou (CN); CENTRAL CHINA SUBCENTER OF NATIONAL CENTER FOR CARDIOVASCULAR DISEASES, Zhengzhou (CN)

(72) Inventors: Yong ZHAO, Zhengzhou (CN); Zhaoyun CHENG, Zhengzhou (CN); Fangyan LIU, Zhengzhou (CN); Yinghui GE, Zhengzhou (CN); Qing LIN, Zhengzhou (CN); Yakun NIU, Zhengzhou (CN); Qingdan YANG, Zhengzhou (CN); Xinlei QI, Zhengzhou (CN)

(73) Assignees: FUWAI CENTRAL CHINA CARDIOVASCULAR HOSPITAL, Zhengzhou (CN); CENTRAL CHINA SUBCENTER OF NATIONAL CENTER FOR CARDIOVASCULAR DISEASES, Zhengzhou (CN)

(21) Appl. No.: 19/025,080

(22) Filed: Jan. 16, 2025

(30) Foreign Application Priority Data
Nov. 8, 2024 (CN) 202411601028.0

(51) Int. Cl. G01N 33/74

(52) U.S. Cl. CPC G01N 33/74; G01N 2333/4753; G01N 2800/32

(57) ABSTRACT

A molecular marker for diagnosing, prognosis, evaluating, and treating cardiac fibrosis and belongs to the technical field of biomedicine. The present disclosure provides a molecular marker capable of diagnosing cardiac fibrosis, assessing the severity of cardiac fibrosis, evaluating the prognosis of cardiovascular disease with cardiac fibrosis, or evaluating the prognosis of acute myocardial infarction. The molecular marker comprises hepatocyte growth factor. It has been found that HGF is significantly elevated in the plasma of patients with cardiac fibrosis and also considerably elevated in patients with aortic stenosis before surgery. Therefore, HGF can be used to diagnose cardiac fibrosis and evaluate its severity.

### TECHNICAL FIELD

The present disclosure relates to a molecular marker for the diagnosis, prognosis evaluation, and treatment of cardiac fibrosis and belongs to the technical field of biomedicine.

### BACKGROUND

Cardiac fibrosis (myocardial fibrosis) is characterized by excessive proliferation of cardiac fibroblasts and excessive extracellular matrix production in myocardial tissue. Under normal circumstances, the extracellular matrix provides mechanical support to myocardial tissue. However, when cardiac fibrosis is excessive or uncontrolled, it can seriously affect the systolic and diastolic function of the heart, as well as the electrophysiological function of the heart. Studies have shown that almost all cardiovascular disease patients have concomitant cardiac fibrosis. Therefore, in clinical practice, the prognosis of a series of major cardiovascular diseases, including hypertension, diastolic heart failure, cardiomyopathy, and sudden cardiac death, can be determined based on the degree of cardiac fibrosis in patients. Research confirms that cardiac fibrosis repair is essential for restoring cardiac function after acute myocardial infarction. Therefore, in clinical practice, the prognosis of acute myocardial infarction can also be judged based on the occurrence of cardiac fibrosis in patients.

Cardiac fibrosis is highly complex and manifests in two aspects: cardiac fibrosis can occur at any stage of cardiovascular disease, and cardiac fibrosis involves multiple cell types. Although fibroblasts are the central executive cells of cardiac fibrosis, other types of cells also play essential roles in the process of cardiac fibrosis. At present, fibrosis is mainly studied by isolating a specific cell type. Myocardial tissue is rich in the dense extracellular matrix, and separating cells through digestion can affect gene expression. Due to these complex factors, there is little in-depth research on cardiac fibrosis. Therefore, at present, there is still a lack of effective treatment methods for cardiac fibrosis, and there is also a lack of suitable intervention methods for restoring cardiac function by controlling cardiac fibrosis after an acute myocardial infarction.

At the same time, the clinical diagnosis of cardiac fibrosis currently relies heavily on invasive tissue biopsy or detection by cardiovascular magnetic resonance late gadolinium enhanced (LGE) and T1 mapping. Wherein invasive tissue biopsy usually involves direct contact with tissues or organs, and patients may experience risks of pain, bleeding, infection, or other complications, while cardiovascular magnetic resonance late gadolinium enhanced (LGE) and T1 mapping detection are complex to operate, require high professional skills from operators, and expensive. If plasma markers directly related to the occurrence and development of cardiac fibrosis can be found, it will be beneficial for the clinical diagnosis of cardiac fibrosis. So far, several plasma markers related to fibrosis have been reported in the literature. Still, these plasma markers are neither derived from the heart nor reflect specific fibrosis stages. Therefore, there is still a lack of plasma markers that can be used for clinical diagnosis of cardiac fibrosis.

### SUMMARY OF THE INVENTION

To address the issues above, the present application provides a molecular marker comprising hepatocyte growth factor (HGF, Scatter Factor), and the molecular marker has any one of the following functions:
(a) diagnosis of cardiac fibrosis (also known as myocardial fibrosis);
(b) evaluation of the severity of cardiac fibrosis;
(c) prognosis evaluation of acute myocardial infarction and
(d) prognosis evaluation of cardiovascular diseases with concomitant cardiac fibrosis other than acute myocardial infarction.

In one embodiment of the present application, cardiac fibrosis comprises cardiac fibrosis caused by pressure load and/or cardiac fibrosis caused by myocardial infarction.

In one embodiment of the present application, the pressure load comprises aortic valve stenosis and/or hypertension.

In one embodiment of the present application, the myocardial infarction comprises acute myocardial infarction.

In one embodiment of the present application, cardiovascular diseases with concomitant cardiac fibrosis other than acute myocardial infarction comprise hypertension, diastolic heart failure, cardiomyopathy, and/or sudden cardiac death.

In one embodiment of the present application, diagnosing cardiac fibrosis involves determining whether a patient has cardiac fibrosis based on the concentration of hepatocyte growth factor in plasma. If the concentration of hepatocyte growth factor in the patient's plasma is much higher than that of the normal population, the patient has cardiac fibrosis. If the concentration of hepatocyte growth factor in the patient's plasma is close to that of the normal population, the patient has no cardiac fibrosis.

In one embodiment of the present application, the prognosis evaluation of acute myocardial infarction comprises judging the patient's prognosis after acute myocardial infarction based on variations in the concentration of hepatocyte growth factor in plasma. If the concentration of hepatocyte growth factor in the patient's plasma temporarily elevates in an immediate transient manner, the patient's prognosis is good. If the concentration of hepatocyte growth factor in the patient's plasma is persistent high-level elevation, persistent low-level elevation, recurrence, or delayed elevation, the patient's prognosis is poor.

The present application further provides the use of a reagent for detecting the molecular marker above in a sample to be tested in the preparation of a product, wherein the product has any one of the following functions:
(a) diagnosis of cardiac fibrosis;
(b) evaluation of the severity of cardiac fibrosis;
(c) prognosis evaluation of acute myocardial infarction and
(d) prognosis evaluation of cardiovascular diseases with concomitant cardiac fibrosis other than acute myocardial infarction.

In one embodiment of the present application, cardiac fibrosis comprises fibrosis caused by pressure load and/or myocardial infarction.

In one embodiment of the present application, the pressure load comprises aortic valve stenosis and/or hypertension.

In one embodiment of the present application, the myocardial infarction comprises acute myocardial infarction.

In one embodiment of the present application, cardiovascular diseases with concomitant cardiac fibrosis other than acute myocardial infarction comprise hypertension, diastolic heart failure, cardiomyopathy, and/or sudden cardiac death.

In one embodiment of the present application, diagnosing cardiac fibrosis involves determining whether a patient has cardiac fibrosis based on the concentration of hepatocyte growth factor in plasma. If the concentration of hepatocyte growth factor in the patient's plasma is much higher than that of the normal population, the patient has cardiac fibrosis. If the concentration of hepatocyte growth factor in the patient's plasma is close to that of the normal population, the patient has no cardiac fibrosis.

In one embodiment of the present application, the prognosis evaluation of acute myocardial infarction comprises judging the patient's prognosis after acute myocardial infarction based on variations in the concentration of hepatocyte growth factor in plasma. If the concentration of hepatocyte growth factor in the patient's plasma temporarily elevates in an immediate transient manner, the patient's prognosis is good. If the concentration of hepatocyte growth factor in the patient's plasma is persistent high-level elevation, persistent low-level elevation, recurrence, or delayed elevation, the patient's prognosis is poor.

In one embodiment of the present application, the product comprises a detection kit.

The present application further provides a product wherein the product comprises a reagent for detecting the molecular marker above in a sample to be tested; the product has any one of the following functions:
(a) diagnosis of cardiac fibrosis;
(b) evaluation of the severity of cardiac fibrosis;
(c) the prognosis evaluation of acute myocardial infarction; and
(d) prognosis evaluation of cardiovascular diseases with concomitant cardiac fibrosis other than acute myocardial infarction.

In one embodiment of the present application, cardiac fibrosis comprises cardiac fibrosis caused by pressure load and/or cardiac fibrosis caused by myocardial infarction.

In one embodiment of the present application, the pressure load comprises aortic valve stenosis and/or hypertension.

In one embodiment of the present application, the myocardial infarction comprises acute myocardial infarction.

In one embodiment of the present application, cardiovascular diseases with concomitant cardiac fibrosis other than acute myocardial infarction comprise hypertension, diastolic heart failure, cardiomyopathy, and/or sudden cardiac death.

In one embodiment of the present application, diagnosing cardiac fibrosis involves determining whether a patient has cardiac fibrosis based on the concentration of hepatocyte growth factor in plasma. If the concentration of hepatocyte growth factor in the patient's plasma is much higher than that of the normal population, the patient has cardiac fibrosis. If the concentration of hepatocyte growth factor in the patient's plasma is close to that of the normal population, the patient has no cardiac fibrosis.

In one embodiment of the present application, the prognosis evaluation of acute myocardial infarction comprises judging the patient's prognosis after acute myocardial infarction based on variations in the concentration of hepatocyte growth factor in plasma. If the concentration of hepatocyte growth factor in the patient's plasma temporarily elevates in an immediate transient manner, the patient's prognosis is good. If the concentration of hepatocyte growth factor in the patient's plasma is persistent high-level elevation, persistent low-level elevation, recurrence, or delayed elevation, the patient's prognosis is poor.

In one embodiment of the present application, the product comprises a detection kit.

The present application further provides a prediction model wherein the prediction model has any one of the following functions:
(a) diagnosis of cardiac fibrosis;
(b) evaluation of the severity of cardiac fibrosis;
(c) prognosis evaluation of acute myocardial infarction; and
(d) prognosis evaluation of cardiovascular diseases with concomitant cardiac fibrosis other than acute myocardial infarction.

The prediction model comprises a detection module, a data analysis module, and a prediction module.

The detection module is used to detect the level of the molecular marker in a sample to be tested.

The data analysis module is used to analyze the detection results output by the detection module.

The prediction module classifies whether the sample to be tested is diseased or has a good or poor prognosis based on the analysis results output by the data analysis module.

In one embodiment of the present application, cardiac fibrosis comprises cardiac fibrosis caused by pressure load and/or cardiac fibrosis caused by myocardial infarction.

In one embodiment of the present application, the pressure load comprises aortic valve stenosis and/or hypertension.

In one embodiment of the present application, the myocardial infarction comprises acute myocardial infarction.

In one embodiment of the present application, cardiovascular diseases with concomitant cardiac fibrosis other than acute myocardial infarction comprise hypertension, diastolic heart failure, cardiomyopathy, and/or sudden cardiac death.

In one embodiment of the present application, diagnosing cardiac fibrosis comprises determining whether a patient has cardiac fibrosis based on the concentration of hepatocyte growth factor in plasma. If the concentration of hepatocyte growth factor in the patient's plasma is much higher than that of the normal population, the patient has cardiac fibrosis. If the concentration of hepatocyte growth factor in the patient's plasma is close to that of the normal population, the patient has no cardiac fibrosis.

In one embodiment of the present application, the prognosis evaluation of acute myocardial infarction comprises judging the patient's prognosis after acute myocardial infarction based on variations in the concentration of hepatocyte growth factor in plasma. If the concentration of hepatocyte growth factor in the patient's plasma temporarily elevates in an immediate transient manner, the patient's prognosis is good. If the concentration of hepatocyte growth factor in the patient's plasma is persistent high-level elevation, persistent low-level elevation, recurrence, or delayed elevation, the patient's prognosis is poor.

In the present application, the term “immediate transient manner” refers to an elevation of HGF levels to more than threefold that of the control within four days after myocardial infarction, followed by a gradual decrease.

The term “persistent low-level elevation” refers to a continuous elevation of HGF levels to 3-10 folds after myocardial infarction for 4 days or more, or there is no day within 7 days that HGF level is more than 2 folds.

The term “persistent high-level elevation” refers to a continuous elevation of HGF levels of more than 10 folds after myocardial infarction for over 3 days.

The term “recurrence” refers to the first elevation of HGF to more than 10 folds after myocardial infarction, followed by another elevation of more than 3 folds after 1-3 days.

The term “delayed elevation” refers to a gradual elevation of more than 7 folds after 6 days of myocardial infarction.

Unless stated otherwise, the terms “subject” and “patient” are used interchangeably in the present application.

The present application further provides a method for preventing or treating cardiac fibrosis, comprising administering one or a combination of hepatocyte growth factor, hepatocyte growth factor signal pathway blocker, and neutrophil infiltration inhibitor to a subject in need. In one embodiment of the present application, cardiac fibrosis comprises cardiac fibrosis caused by pressure load, cardiac fibrosis induced by myocardial infarction, or cardiac fibrosis concomitant with cardiovascular diseases other than acute myocardial infarction.

In one embodiment of the present application, cardiovascular diseases other than acute myocardial infarction comprise hypertension, diastolic heart failure, cardiomyopathy, and/or sudden cardiac death.

In one embodiment of the present application, when cardiac fibrosis is caused by myocardial infarction, the method comprises administering a hepatocyte growth factor to a subject experiencing acute myocardial infarction or administering a hepatocyte growth factor signal pathway blocker and/or a neutrophil infiltration inhibitor to a subject experiencing acute myocardial infarction.

In one embodiment of the present application, when cardiac fibrosis is concomitant with cardiovascular diseases other than acute myocardial infarction, the method comprises administering a hepatocyte growth factor and/or hepatocyte growth factor signal pathway blocker to the subject experiencing cardiac fibrosis.

In one embodiment of the present application, the method comprises:
if the plasma hepatocyte growth factor level in a subject experiencing acute myocardial infarction after treatment is persistent high-level elevation or recurrence, administering hepatocyte growth factor signal pathway blocker and/or neutrophil infiltration inhibitor to the subject;
if the plasma hepatocyte growth factor level in a subject experiencing acute myocardial infarction after treatment is insufficient (for example, HGF persistent low-level elevation), administering a hepatocyte growth factor to the subject and
if the plasma hepatocyte growth factor level in a subject experiencing acute myocardial infarction after treatment is delayed elevation, administering a hepatocyte growth factor signal pathway blocker to the subject to adjust the activation time of the hepatocyte growth factor signal pathway to improve the subject's prognosis.

In one embodiment of the present application, when an acute myocardial infarction occurs, the method comprises administering hepatocyte growth factor to the subject or administering a hepatocyte growth factor signal pathway blocker and/or neutrophil infiltration inhibitor to the subject to control fibroblast proliferation dominated by an acute inflammatory response and/or control myofibroblast-mediated fibrosis repair.

In one embodiment of the present application, the hepatocyte growth factor signal pathway blocker comprises one or a combination of a hepatocyte growth factor inhibitor, an AKT inhibitor, a mTORC1 inhibitor, and a MET inhibitor.

The MET inhibitors include but are not limited to JNJ-38877605, Cabozantinib, and SAR125844.

The AKT inhibitors include but are not limited to Capivasertib, GCD-0068, and Afuresertib.

The mTORC1 inhibitors include but are not limited to rapamycin, Everolimus, and AZD-8055.

The HGF inhibitors include but are not limited to Erlotinib, Cetuximab, and HGF monoclonal antibodies. HGF monoclonal antibodies such as Ficlatuzumab.

In one embodiment of the present application, the neutrophil infiltration inhibitor comprises erythromycin.

In one embodiment of the present application, the dose of HGF is 0.6 to 2.4 mg/m²/day, administered intravenously continuously for 12 to 14 days, wherein m² refers to the surface area of the human body.

In one embodiment of the present application, the dose of the HGF signal pathway blocker or neutrophil infiltration inhibitor is 10 mg to 4 g per day, or 60 mg to 4 g per day, or 1 g to 4 g per day, or 10 to 60 mg per day, 60 mg to 1 g per day.

In practical applications, the actual dose may be related to the severity of the condition.

In one embodiment of the present application, the dose range of recombinant HGF is 0.6 to 2.4 mg/m²/day, administered intravenously continuously for 12 to 14 days. (m² refers to the surface area of the human body).

In one embodiment of the present application, when the MET inhibitor is JNJ-38877605, the administration method is oral, and the dose range is from 10 to 60 mg per day.

In one embodiment of the present application, when the neutrophil infiltration inhibitor is erythromycin, the dose range is from 250 mg to 1 g every 6 hours (i.e., between 1 g and 4 g per day).

The present application further provides a method for diagnosing and/or evaluating cardiac fibrosis; the method comprises:

Determining whether a patient has cardiac fibrosis based on the concentration of HGF in plasma. If the concentration of HGF in the patient's plasma is much higher than that of the normal population, it is determined that the patient has cardiac fibrosis. If the concentration of HGF in the patient's plasma is close to that of the normal population, it is determined that the patient has no cardiac fibrosis.

The present application further provides a method for evaluating the prognosis of acute myocardial infarction; the method comprises:

Judging the patient's prognosis after acute myocardial infarction based on variations in the concentration of hepatocyte growth factor in plasma, if the concentration of hepatocyte growth factor in the patient's plasma temporarily elevates in an immediate transient manner, the patient's prognosis is good. If the concentration of hepatocyte growth factor in the patient's plasma is persistent high-level elevation, persistent low-level elevation, recurrence, or delayed elevation, the patient's prognosis is poor.

The present application further provides a method for improving the prognosis of patients with acute myocardial infarction; the method comprises:
1) evaluating the prognosis of acute myocardial infarction, comprising judging the prognosis of the patient after treatment based on variations in the concentration of hepatocyte growth factor in plasma, if the concentration of hepatocyte growth factor in the patient's plasma temporarily elevations in an immediate transient manner, the patient's prognosis is good; and if the concentration of hepatocyte growth factor in the patient's plasma is persistent high-level elevation, persistent low-level elevation, recurrence or delayed elevation, the patient's prognosis is poor; and
2) administering a hepatocyte growth factor, a hepatocyte growth factor signal pathway blocker, or a neutrophil infiltration inhibitor to patients with poor prognosis, comprising:
when the hepatocyte growth factor level in patients after treatment is persistent high-level elevation or recurrence, administering hepatocyte growth factor signal pathway blocker or neutrophil infiltration inhibitor to the subject; when the plasma hepatocyte growth factor level in patients with acute myocardial infarction after treatment is persistent low-level elevation, administering a hepatocyte growth factor to the patient; and when the hepatocyte growth factor level in patients after treatment delayed elevation, administering a hepatocyte growth factor signal pathway blocker to the patient to adjust the activation time of the hepatocyte growth factor signal pathway to improve the patient's prognosis.

In one embodiment of the present application, the hepatocyte growth factor signal pathway blocker comprises one or a combination of a hepatocyte growth factor inhibitor, an AKT inhibitor, a mTORC1 inhibitor, and a MET inhibitor.

The MET inhibitors include but are not limited to JNJ-38877605, Cabozantinib, and SAR125844.

The AKT inhibitors include but are not limited to Capivasertib, GCD-0068, and Afuresertib.

The mTORC1 inhibitors include but are not limited to rapamycin, Everolimus, and AZD-8055.

The HGF inhibitors include but are not limited to Erlotinib, Cetuximab, and HGF monoclonal antibodies. HGF monoclonal antibodies such as Ficlatuzumab.

In one embodiment of the present application, the neutrophil infiltration inhibitor comprises erythromycin.

In one embodiment of the present application, the dose of hepatocyte growth factor is 0.6 to 2.4 mg/m²/day, administered intravenously continuously for 12 to 14 days, wherein m² refers to the surface area of the human body;

In one embodiment of the present application, the dose of the hepatocyte growth factor signal pathway blocker or neutrophil infiltration inhibitor is 10 mg to 4 g per day, or 60 mg to 4 g per day, or 1 g to 4 g per day, or 10 to 60 mg per day, 60 mg to 1 g per day.

In practical applications, the actual dose may be related to the severity of the condition.

In one embodiment of the present application, the dose range of recombinant HGF is 0.6 to 2.4 mg/m²/day, administered intravenously continuously for 12 to 14 days. (m² refers to the surface area of the human body).

In one embodiment of the present application, when the MET inhibitor is JNJ-38877605, the administration method is oral, and the dose range is from 10 to 60 mg per day.

In one embodiment of the present application, when the neutrophil infiltration inhibitor is erythromycin, the dose range is from 250 mg to 1 g every 6 hours (i.e., between 1 g and 4 g per day).

The present application further provides a method for screening drugs. The drugs comprise drugs for treating cardiac fibrosis, acute myocardial infarction, and/or cardiovascular diseases with concomitant cardiac fibrosis other than acute myocardial infarction.

The method comprises administering the drug to be screened to the experimental subject, obtaining the results related to the molecular markers above after administering the drug to be screened to the experimental subjects, and determining whether the drug to be screened is effective or ineffective based on the obtained results.

The present application provides the use of hepatocyte growth factor, hepatocyte growth factor signal pathway blocker, and/or neutrophil infiltration inhibitor in the preparation of a drug, wherein the drug has any one of the following functions:
(a) prevention and/or treatment of cardiac fibrosis;
(b) prevention and/or treatment of acute myocardial infarction and
(c) prevention and/or treatment of cardiovascular diseases with concomitant cardiac fibrosis other than acute myocardial infarction.

In one embodiment of the present application, cardiac fibrosis comprises cardiac fibrosis caused by pressure load and/or cardiac fibrosis caused by myocardial infarction.

In one embodiment of the present application, the pressure load comprises aortic valve stenosis and/or hypertension.

In one embodiment of the present application, the myocardial infarction comprises acute myocardial infarction.

In one embodiment of the present application, cardiovascular diseases with concomitant cardiac fibrosis other than acute myocardial infarction comprise hypertension, diastolic heart failure, cardiomyopathy, and/or sudden cardiac death.

In one embodiment of the present application, when the drug has the function of prevention and/or treatment of cardiac fibrosis or cardiovascular diseases with concomitant cardiac fibrosis other than acute myocardial infarction, the components of the drug comprise hepatocyte growth factor and/or hepatocyte growth factor signal pathway blocker.

In one embodiment of the present application, when the drug prevents and/or treats acute myocardial infarction, its components comprise hepatocyte growth factor, hepatocyte growth factor signal pathway blocker, and/or neutrophil infiltration inhibitor.

In one embodiment of the present application, treating acute myocardial infarction comprises controlling fibroblast proliferation dominated by an acute inflammatory response and/or controlling myofibroblast-mediated fibrosis repair. The specific methods comprise administering HGF, MET inhibitors, AKT inhibitors, mTORC1 inhibitors, and neutrophil infiltration inhibitors.

In one embodiment of the present application, the hepatocyte growth factor signal pathway blocker comprises one or a combination of a hepatocyte growth factor inhibitor, an AKT inhibitor, a mTORC1 inhibitor, and a MET inhibitor.

The MET inhibitors include but are not limited to JNJ-38877605, Cabozantinib, and SAR125844.

The AKT inhibitors include but are not limited to Capivasertib, GCD-0068, and Afuresertib.

The mTORC1 inhibitors include but are not limited to rapamycin, Everolimus, and AZD-8055.

The HGF inhibitors include but are not limited to Erlotinib, Cetuximab, and HGF monoclonal antibodies. HGF monoclonal antibodies such as Ficlatuzumab. In one embodiment of the present application, the neutrophil infiltration inhibitor comprises erythromycin.

The present application further provides a drug wherein the drug has any one of the following functions:
(a) prevention and/or treatment of cardiac fibrosis;
(b) prevention and/or treatment of acute myocardial infarction and
(c) prevention and/or treatment of cardiovascular diseases with concomitant cardiac fibrosis other than acute myocardial infarction.

The drug's components comprise hepatocyte growth factor, hepatocyte growth factor signal pathway blocker, and/or neutrophil infiltration inhibitor.

In one embodiment of the present application, cardiac fibrosis comprises cardiac fibrosis caused by pressure load and/or cardiac fibrosis caused by myocardial infarction.

In one embodiment of the present application, the pressure load comprises aortic valve stenosis and/or hypertension.

In one embodiment of the present application, the myocardial infarction comprises acute myocardial infarction.

In one embodiment of the present application, cardiovascular diseases with concomitant cardiac fibrosis other than acute myocardial infarction comprise hypertension, diastolic heart failure, cardiomyopathy, and/or sudden cardiac death.

In one embodiment of the present application, when the drug has the function of prevention and/or treatment of cardiac fibrosis or cardiovascular diseases with concomitant cardiac fibrosis other than acute myocardial infarction, the components of the drug comprise hepatocyte growth factor and/or hepatocyte growth factor signal pathway blocker.

In one embodiment of the present application, when the drug prevents and/or treats acute myocardial infarction, its components comprise hepatocyte growth factor, hepatocyte growth factor signal pathway blocker, and/or neutrophil infiltration inhibitor.

In one embodiment of the present application, treating acute myocardial infarction comprises controlling fibroblast proliferation dominated by an acute inflammatory response and/or controlling myofibroblast-mediated fibrosis repair. The specific methods comprise administering HGF, MET inhibitors, AKT inhibitors, mTORC1 inhibitors, and neutrophil infiltration inhibitors. In one embodiment of the present application, the hepatocyte growth factor signal pathway blocker comprises one or a combination of a hepatocyte growth factor inhibitor, an AKT inhibitor, a mTORC1 inhibitor, and a MET inhibitor.

The MET inhibitors include but are not limited to JNJ-38877605, Cabozantinib, and SAR125844.

The AKT inhibitors include but are not limited to Capivasertib, GCD-0068, and Afuresertib.

The mTORC1 inhibitors include but are not limited to rapamycin, Everolimus, and AZD-8055.

The HGF inhibitors include but are not limited to Erlotinib, Cetuximab, and HGF monoclonal antibodies. HGF monoclonal antibodies such as Ficlatuzumab.

In one embodiment of the present application, the neutrophil infiltration inhibitor comprises erythromycin.

The technical solution of the present application has the following advantages:
1. The present application provides a molecular marker comprising hepatocyte growth factor (HGF, Scatter Factor); the molecular marker has any one of the following functions: (a) diagnosis of cardiac fibrosis; (b) evaluation of the severity of cardiac fibrosis; (c) prognosis evaluation of cardiovascular diseases with concomitant cardiac fibrosis; and (d) prognosis evaluation of acute myocardial infarction.

The inventor of the present application has found that HGF levels are significantly elevated in the plasma of patients with cardiac fibrosis, and in patients with aortic valve stenosis before surgery, plasma HGF levels are also significantly elevated. Therefore, HGF can be used to diagnose cardiac fibrosis and evaluate its severity. At the same time, patients with cardiovascular disease often have concomitant cardiac fibrosis, and excessive cardiac fibrosis in cardiovascular disease patients generally has a poor prognosis. Therefore, HGF can also be used to evaluate the prognosis of cardiovascular disease with concomitant cardiac fibrosis.

The repair of cardiac injury after acute myocardial infarction comprises two stages: acute inflammatory response and fibrosis repair. Where acute inflammatory response generally occurs in the early stage of the cardiac injury repair process, while fibrosis repair typically occurs in the late stage or after the end of the acute inflammatory response. Research has found that for acute inflammatory reactions in the heart, the level of HGF in the plasma of patients with acute myocardial infarction positively correlates with the activation status of neutrophils in patients with acute myocardial infarction. The activation status of neutrophils can be used to determine whether immediate and effective acute inflammation has occurred in patients with acute myocardial infarction. For the overall repair of cardiac injury after acute myocardial infarction having two stages of acute inflammatory response and fibrosis repair, immediate transient elevation of HGF in the plasma of patients with acute myocardial infarction significantly correlates with good prognosis. Persistent low-level elevation, persistent high-level elevation, recurrence, and delayed elevation of HGF are significantly associated with poor prognosis. In contrast, a good prognosis means that patients with acute myocardial infarction can immediately and effectively repair cardiac injury after treatment and restore heart function.

In contrast, a poor prognosis means that patients with acute myocardial infarction cannot immediately and effectively repair cardiac injury and then restore heart function after receiving treatment. Therefore, HGF can be used to evaluate the prognosis of acute myocardial infarction. The term “immediate and effective” refers to fibrosis repair occurring within 7 days after treatment in patients with acute myocardial infarction, which also means a good prognosis.
2. The present application provides the use of hepatocyte growth factor, hepatocyte growth factor signal pathway blocker, and/or neutrophil infiltration inhibitor in the preparation of a drug, wherein the drug has any one of the following functions: (a) prevention and/or treatment of cardiac fibrosis; (b) prevention and/or treatment of cardiovascular diseases with concomitant cardiac fibrosis; and (c) prevention and/or treatment of acute myocardial infarction.

The repair of cardiac injury after acute myocardial infarction comprises two stages: acute inflammatory response and fibrosis repair. Where acute inflammatory response generally occurs in the early stage of the cardiac injury repair process, while fibrosis repair typically occurs in the late stage or after the end of the acute inflammatory response. The inventor of the present application found that the elevated plasma HGF after myocardial infarction originates from neutrophils, not cardiac fibroblasts. The HGF-MET signal does not affect the migration and infiltration of neutrophils into myocardial tissue after myocardial infarction; inhibition of neutrophil tissue infiltration can inhibit the inflammatory response of the injured tissue. Moreover, inhibiting the inflammatory response of injured tissues is not beneficial to the repair of cardiac injury after acute myocardial infarction. It can be seen that the process of neutrophils releasing HGF is involved in the proliferation of fibroblasts dominated by acute inflammatory response after acute myocardial infarction. The inventor of the present application has found that the HGF signal pathway is crucial for the proliferation of fibroblasts dominated by the acute inflammatory response after acute myocardial infarction. The inventor has found that HGF can significantly promote fibroblast proliferation but inhibit the collagen synthesis function of myofibroblasts. Inhibition of MET or mTORC1 signal will block the effect of HGF on promoting fibroblast proliferation but promote fibrosis of myofibroblasts. It can be seen that the HGF-MET signal can promote the proliferation of cardiac fibroblasts after myocardial infarction but inhibit the collagen synthesis function of myofibroblasts (i.e., inhibit fibrosis).

The HGF signal pathway is crucial in the fibrosis repair mediated by myofibroblasts after acute myocardial infarction. Therefore, in cardiac fibrosis and acute myocardial infarction, the different roles of the HGF signal pathway in neutrophil-dominated fibroblast proliferation and myofibroblast-mediated extracellular matrix synthesis can be regulated based on plasma HGF and its dynamic variations to treat cardiac fibrosis and control effectively injure repair after acute myocardial infarction. HGF, HGF signaling pathway blockers, and/or neutrophil infiltration inhibitors and compounds that can regulate the HGF signal pathway and neutrophil release of HGF can be used to develop drugs for treating cardiac fibrosis and acute myocardial infarction. For example, if the HGF level in patients with acute myocardial infarction is persistent high-level elevation or recurrence after treatment, hepatocyte growth factor signal pathway blockers and/or neutrophil infiltration inhibitors can be administered to improve the patient's prognosis. If the HGF level of patients with acute myocardial infarction is insufficient after treatment (for example, if the HGF level is persistent low-level elevation), HGF can be supplemented to improve the patient's prognosis. Suppose patients with acute myocardial infarction experience delayed elevation of HGF after treatment. In that case, the HGF signal pathway activation time can be adjusted by administering hepatic growth factor signal pathway blockers to improve patient prognosis.

### DESCRIPTION OF THE DRAWINGS

This patent or application file contains at least one drawing executed in color. Copies of this patent or patent application publication with color drawing(s) will be provided by the office upon request and payment of the necessary fee.

FIG. 1: WGCNA analysis flowchart. Eight-time points spanning acute and chronic phases were selected based on LCA and TAC models.

FIG. 2: The brown module is related to cardiac mechanical stress and cardiac muscle cell contraction. In the brown module, enrichment is calculated relative to random expectations (relative to mice transcriptome). Enrichment factor 1 indicates no enrichment. The p-value was calculated based on the hypergeometric distribution.

FIG. 3: Turquoise, pink, and yellow modules are associated with neutrophils/proliferating fibroblasts, focal adhesion, and myofibroblasts.

FIG. 4: Different dynamic characteristics of turquoise, pink, and yellow modules during cardiac fibrosis. Genes from different modules with up-regulated genes from cardiac fibroblasts at Day 3, Day 7, Week 2, and Week 4 after myocardial infarction were compared, and enrichment levels were calculated.

FIG. 5: Plasma HGF significantly elevated in a mice model of acute myocardial infarction. Plasma HGF peaked on day 3 after myocardial infarction but decreased on day 7 after myocardial infarction. After myocardial infarction surgery, erythromycin was injected intraperitoneally every day, and HGF levels were measured on day 3.

FIG. 6: On day 3 after myocardial infarction, cardiac fibroblasts did not elevate HGF secretion. Cardiac fibroblasts were isolated from sham surgery and MI mice models and cultured in a 2 mL medium for 4 hours, followed by measurement of HGF levels.

FIG. 7: Neutrophils secrete HGF under pro-inflammatory stimulation. Neutrophils were isolated from peripheral blood, cultured in medium, and treated with serum amyloid protein a (10 μg/mL) for 5 hours, followed by measurement of HGF levels.

FIG. 8: CEBPδ controls the production of HGF by neutrophils. Neutrophils were isolated from the peripheral blood of Cebpδ knockdown mice, cultured in culture medium, and treated with serum amyloid a (10 μg/mL) for 5 hours, followed by measurement of HGF levels.

FIG. 9: HGF does not affect the infiltration of neutrophils in myocardial tissue after acute myocardial infarction. White blood cells and neutrophils were isolated from the left ventricle of mice with acute myocardial infarction and acute myocardial infarction plus JNJ treatment, and the proportion of neutrophils to white blood cells was calculated. White blood cells and neutrophils (CD45+CD11b+Ly6G+) were separated using BD FACSARIA III flow cytometry.

FIG. 10: TNF superfamily ligands and their receptors that induce non-canonical NF-κB are enriched in turquoise instead of the yellow module.

FIG. 11: TNFR2 interaction proteins are associated with turquoise modules and proliferating fibroblasts. The interaction proteins of TNFR2 were sourced from the GPS protein interaction database and compared with up-regulated genes from cardiac fibroblasts on Day 3, Day 7, Week 2, and Week 4 after myocardial infarction.

FIG. 12: Colocalization of TNFα and TNFR2 in the myocardial tissue membrane on day 3after myocardial infarction. Red: TNFα signal, green: TNFR2 signal. Scale bar: 25 μm.

FIG. 13: NF-κB2 (nuclear green signal) significantly elevated in cardiac fibroblasts on day 3 after myocardial infarction, and ACTA2 (red) labeled cardiac fibroblasts. Scale bar: 25 μm.

FIG. 14: Neutrophil membrane TNFα promotes cardiac fibroblast proliferation through NF-κB2. Electroporation may affect the co-culture of fibroblasts and neutrophils. Therefore, the percentage of EdU-positive fibroblasts decreased in all three groups of cells undergoing electroporation.

FIG. 15: Neutrophil membrane TNFα activates non-canonical NF-κB2 in cardiac fibroblasts. Cultivate fibroblasts separately or co-culture them with neutrophils and treat them with TACE inhibitor TMI-1 or siRNA targeting NF-κB2. Scale bar: 50 μm.

FIG. 16: Non-canonical NF-κB2 activates the expression of MET in fibroblasts. Cultivate fibroblasts and neutrophils together. Wash away neutrophils and purify fibroblasts for protein blotting.

FIG. 17: The HGF-MET signal pathway is activated in myocardial tissue of acute myocardial infarction. Cardiac fibroblasts were isolated and cultured from mice with sham surgery and myocardial infarction models. Fibroblasts were treated with HGF and MET inhibitors. Blotting of fibroblasts was performed using antibodies against MET and MET phosphate (Y1234/Y1235).

FIG. 18: The HGF-MET signal pathway is activated in the myocardial tissue of the TAC mice model. Cardiac fibroblasts were isolated from mice with sham surgery and TAC models. Blotting of fibroblasts was performed using antibodies against MET and MET phosphate (Y1234/Y1235).

FIG. 19: HGF promotes the proliferation of cardiac fibroblasts. Fibroblasts were isolated from myocardial tissue on day 5 after MI, cultured in vitro, and then treated with HGF, MET inhibitors, and rapamycin. The number of viable cells was measured using a sensitive colorimetric assay to compare cell proliferation.

FIG. 20: Analysis of differentially expressed genes after HGF treatment. The turquoise module and mTORC1 signal-related genes were enriched in genes up-regulated after HGF treatment. The genes downregulated after HGF treatment are enriched in the yellow module and EPRS1 target.

FIG. 21: The target of EPRS1 is enriched in the yellow module and core matrisome (extracellular matrix protein) but not in the turquoise module. The enrichment analysis results are consistent with the role of EPRS1 in ECM protein synthesis.

FIG. 22: HGF activates the AKT-mTORC1 pathway in cardiac fibroblasts. Fibroblasts were isolated from the left ventricle of mice on day 3 after myocardial infarction, cultured, and treated with HGF and rapamycin. Immunofluorescence staining was performed using antibodies targeting activated AKT and p70 S6 kinase forms. Scale bar: 25 μm.

FIG. 23: In a mice model of myocardial infarction, MET-mediated signal activates the AKT-mTORC1 pathway in cardiac fibroblasts. IHC was performed on left ventricular tissue on day 3 after myocardial infarction. Scale bar: 50 μm.

FIG. 24: In the TAC mice model, the MET-mediated pathway activates the AKT-mTORC1 pathway in cardiac fibroblasts. IHC was performed on the left ventricular tissue on day 5 after TAC surgery. Scale bar: 50 μm.

FIG. 25: The MET-AKT-mTORC1 pathway promotes the proliferation of cardiac fibroblasts in a mice model of myocardial infarction. Perform IHC on left ventricular tissue on day 3 after myocardial infarction. Scale bar: 50 μm.

FIG. 26: The MET-AKT-mTORC1 pathway promotes the proliferation of cardiac fibroblasts in the TAC mice model. Perform IHC on the left ventricular tissue on day 5 after TAC surgery. Scale bar: 50 μm.

FIG. 27: The HGF-AKT-mTORC1-EPRS1 pathway inhibits TGFβ1-induced fibroblast differentiation. On day 3 after myocardial infarction, cardiac fibroblasts were isolated from the left ventricle, cultured, and treated with TGFβ1, HGF, JNJ (MET inhibitor), RAP (rapamycin, mTORC1 inhibitor), and Halofuginone (EPRS1 inhibitor). Immunofluorescence staining was performed using antibodies against ACTA2 to display fibroblasts. Scale bar: 50 μm.

FIG. 28: During TGFβ1-induced fibroblast differentiation, the HGF-AKT-mTORC1-EPRS1 pathway inhibits collagen synthesis. On day 3 after myocardial infarction, cardiac fibroblasts were isolated from the left ventricle, cultured, and treated as described in FIG. 28, and hydroxyproline content was measured. Halofuginone, HAL.

FIG. 29: Activated mTORC1 promotes phosphorylation of EPRS1. Cardiac fibroblasts were isolated from the left ventricle on day 7 after myocardial infarction, cultured for 4 hours, and treated with HGF and rapamycin. Western blot analysis was performed using antibodies targeting EPRS1 and its phosphorylation.

FIG. 30: The MET-AKT-mTORC1 pathway inhibits collagen synthesis in the heart of a myocardial infarction mice model. MET inhibitor and rapamycin were treated from day 3 to day 10 after myocardial infarction. HGF (250 μg/kg) was injected from day 4 to day 7 after myocardial infarction to determine the hydroxyproline content in the left ventricle.

FIG. 31: The MET-AKT-mTORC1 pathway inhibits collagen synthesis in the hearts of TAC mice models. From day 7 to day 15 after TAC, the left ventricular hydroxyproline content was measured by treating with MET inhibitors and rapamycin.

FIG. 32: Preoperative elevated plasma HGF levels in patients with myocardial fibrosis and aortic stenosis.

FIG. 33: Dynamic cardiac troponin T and HGF changes after acute myocardial infarction. Within 2 days after acute myocardial infarction, there is a sharp elevation in troponin T in the blood. This study monitored the dynamic variations of HGF levels in 46 hospitalized patients with acute myocardial infarction (including ST elevation and non-ST elevation) and found that HGF dynamic variations can be divided into five categories: immediate transient elevation, persistent low-level elevation, persistent high-level elevation, recurrence, and delayed elevation.

FIG. 34: The dynamic variations of HGF after acute myocardial infarction were significantly correlated with the prognosis of patients with acute myocardial infarction during hospitalization. Among 46 patients with acute myocardial infarction, 17 patients in the immediate transient elevation group, 15 patients had a good prognosis, and only two patients had a poor prognosis. In contrast, all 18 patients in the continuously elevated, recurrent, and delayed repair groups had poor prognoses (Chi-square test, p-value 4.066e-07).

FIG. 35: Correlation between HGF and clinical biochemical indicators (i.e., NLR, BNP, creatine kinase myocardial band (CK-MB) volume and activity, and hs-cTnT) measured after acute myocardial infarction (AMI). The correlation among indicators was calculated by the Spearman method, and the corresponding P-values were in square brackets under the correlation coefficient.

FIG. 36: Inflammation and HGF signal intensity determine the prognosis of acute myocardial infarction. In the left coronary artery permanent ligation (LCA) induced model of acute myocardial infarction, the survival rate was approximately 78% (N=3 groups, total 28). When MET inhibitor JNJ was administered after acute myocardial infarction, the survival rate was approximately 32% (N=3 groups, total 28). After acute myocardial infarction, erythromycin was administered, and the survival rate was about 35% (N=3 groups, total 28). After acute myocardial infarction, HGF was administered, and the survival rate was approximately 46% (N=3 groups, total of 25).

### DETAILED DESCRIPTION

The following examples are provided to help readers better understand the present application. They are not limited to the best embodiments and do not limit the content or protection scope of the present application. Any product identical or similar to the present application obtained by combining its features with those of other prior art shall fall within the protection scope of the present application.

Some specific experimental steps or conditions not indicated in the examples can be carried out according to the operation or conditions of conventional experimental steps described in the literature in this field. If the manufacturer of the reagent or instrument used is not indicated, it is a traditional product of reagent commercially available.

The MET inhibitor used in the following experimental examples of the present application is JNJ-38877605 (purchased from MedChemExpress, item number HY-50683, using 0.1 mL PBS buffer as the solvent).

### Experimental Example 1

The experimental process is as follows:

### 1. Use WGCNA Analysis to Identify Three Gene Groups Related to Cardiac Fibrosis

Myocardial infarction (MI) and pressure load are probably the two most common causes of cardiac fibrosis. Myocardial infarction is one of the most common causes of heart failure and can lead to reparative fibrosis. Hypertension or valvular disease can lead to pressure overload and reactive or diffuse myocardial fibrosis. In this study, the myocardial infarction model of left coronary artery permanent ligation (LCA) and the transverse aortic constriction (TAC) model were used as two representative and relevant models to study cardiac fibrosis.

Eighty-eight C57BL/6J mice (purchased from Beijing Huafukang Laboratory Animal Company, about 8 weeks old) were used. Refer to X for “Curaj A, et al. J Vis Exp. 2015 May 4: (99):e52197.” and “Zaw AM, et al. J Vis Exp. 2017 Mar 14:(121):55293.”, myocardial infarction mice model (MI mice model) obtained by left coronary artery permanent ligation (LCA). A transverse aortic constriction (TAC mice model) was obtained by transverse aortic constriction (TAC) surgery for subsequent study.

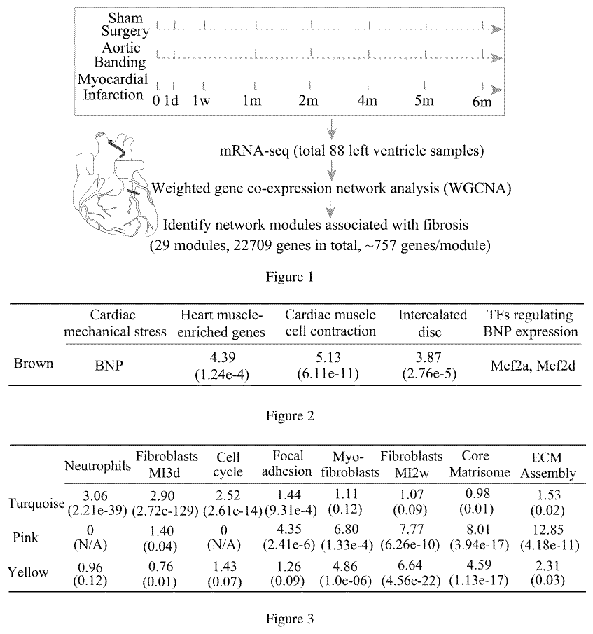

This study, 8 time points were selected to study the dynamic process of cardiac fibrosis, including the acute and chronic stages. Wherein the acute phase included pre-surgery (i.e., day 0), Day 1, and Week 1 after acute myocardial infarction and TAC surgery, and the chronic phase included Month 1, Month 2, Month 4, Month 5, and Month 6 after the acute myocardial infarction and TAC surgery. To study cardiac fibrosis, weighted gene co-expression network analysis (WGCNA) was used to reveal the shared mechanism of cardiac fibrosis. WGCNA divided the murine transcriptome of 22,709 genes into 29 gene groups, averaging 757 genes per gene group (FIG. 1).

First, this study assessed whether the classified gene groups reflected actual cardiac biology. Brain natriuretic peptide (BNP) encoded by NPPB is specifically expressed in cardiac muscle cells. BNP is a recognized biomarker of cardiac mechanical stress, and elevated BNP is commonly used to diagnose heart failure. NPPB is clustered into brown modules (or gene clusters). In this study, the published cardiac muscle cell enrichment gene set was used (for published cardiac muscle cell enrichment gene set, refer to “Uhlen M et al. Science. 2015 Jan. 23; 347(6220):1260419.”), and compared this myocardial concentration with the Brown module. Myocardium-rich gene groups are highly enriched in the brown module, indicating that the Brown module is mainly composed of genes derived from cardiac muscle cells (FIG. 2). Genes associated with cardiac muscle cell contraction are abundant in the Brown module. Intercalated discs (ID) of myocardial tissue connect adjacent cardiac muscle cells and provide mechanosensitivity. The Brown module also highly enriches the ID component gene (FIG. 2). These analyses strongly suggest that the Brown module comprises genes from cardiac muscle cells and senses mechanical stress. Mef2a and Mef2d transcription factors directly activating BNP expression are clustered in the Brown module (FIG. 2). These data show that WGCNA correctly clusters functionally related genes into the same module or gene group.

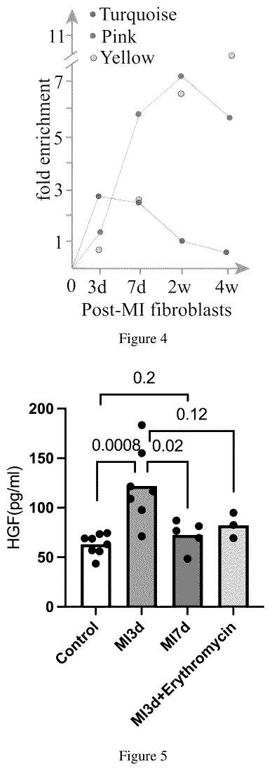

This study then identified which modules were associated with cardiac fibroblasts. On day 3 after myocardial infarction, cardiac fibroblasts were mostly proliferative, while cardiac tissue was rich in myofibroblasts on Week 2 after myocardial infarction. In this study, the up-regulated genes of fibroblasts on day 3 or week 2 after myocardial infarction were used for enrichment analysis. Turquoise module (5415) was associated with fibroblasts on day 3 after myocardial infarction, and pink (435) and yellow (1319) modules were associated with myofibroblasts (FIG. 3). After myocardial infarction, neutrophils infiltrate the myocardial tissue and peak around day 3. Neutrophil-specific genes and cell cycle-related genes were enriched in the turquoise module. These analyses showed that the turquoise modules were involved in the proliferation of neutrophils and fibroblasts. Myofibroblasts secrete high levels of extracellular matrix, such as collagen, compared with proliferating fibroblasts. Consistent with the up-regulated genes observed two weeks after myocardial infarction, genes related to the extracellular matrix or extracellular matrix assembly in the core were significantly enriched in pink and yellow modules (FIG. 3). These analyses highly suggested that the pink and yellow modules were associated with myofibroblasts. This study evaluated the dynamic changes of these modules during the progression of cardiac fibrosis by comparing the up-regulated genes from cardiac fibroblasts at day 3, day 7, week 2, and week 4 after myocardial infarction (FIG. 4). Turquoise modules peaked on day 3 after myocardial infarction and gradually decreased. Pink modules peaked 2 weeks after myocardial infarction and declined thereafter. The yellow module remained active even four weeks after myocardial infarction.

Based on these analyses, the present study concluded that the turquoise module contains genes associated with acute inflammation and fibroblast proliferation, and the yellow module contains genes related to myofibroblasts.

### 2. Essential Roles of Neutrophil Release of HGF Growth Factor for Cardiac Repair after Acute Myocardial Infarction

Various growth factor families, such as PDGF, FGF, EGF, and HGF (hepatocyte growth factor), are grouped into turquoise modules. The role of PDGF and FGF families in regulating fibroblast proliferation has been well studied. Among these growth factors, HGF and its receptor, MET, are also in the turquoise module, but the HGF-MET signal pathway has not been studied in cardiac fibrosis. This study used a myocardial infarction model with left coronary artery permanent ligation (LCA) and a transverse aortic constriction (TAC mice model) to investigate the role of the HFG-MET signal pathway in cardiac fibrosis.

This study first identified the dynamic variations of HGF after acute myocardial infarction. Twelve mice with myocardial infarction obtained by LCA were selected as the model group. Three mice with myocardial infarction obtained by LCA were chosen as the experimental group, which were administered by intraperitoneal injection of erythromycin (purchased from MedChemExpress, article No. HY-B 0220, with 0.1 mL PBS buffer as solvent) at a dose of 100 mg/kg mice weight daily after myocardial infarction surgery. The intervention was performed once a day from day 1 to day 7 after myocardial infarction surgery. During the intervention period, the plasma HGF level of mice was detected by ELISA every day using sham operation mice as controls. The results showed that plasma HGF in the model group increased significantly on Day 3 after myocardial infarction but decreased on Day 7 after myocardial infarction (FIG. 5). Moreover, erythromycin intervention significantly reduced the elevation of plasma HGF in 3 days after myocardial infarction in the experimental group compared to the model group (FIG. 5).

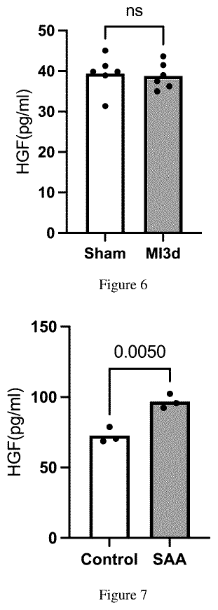

In this study, cardiac fibroblasts were isolated from mice on day 3 after myocardial infarction, and their secreted HGF levels were measured. Six mice with myocardial infarction were obtained, and the sham operation mice were used as controls. Cardiac fibroblasts were isolated from the mice on day 3 after acute myocardial infarction (i.e., on day 3 after myocardial infarction surgery) (for isolation methods, refer to “Pramod Sahadevan, Bruce G Allen, Methods. 2022 July; 203:187-195.”). Fibroblasts were inoculated with 2×10⁵ cells into 2 ml DMEM/F-12 serum-free medium (purchased from Gibco) supplemented with 10% (v/v) fetal bovine serum (purchased from Gibco) and then cultured in a sterile cell incubator at 37° C. and 5% (v/v) CO₂ for 4 hours. After the culture, the culture supernatant was taken, and ELISA detected the HGF level secreted by cardiac fibroblasts. The measurement results showed no difference in HGF between the myocardial infarction and sham surgery groups, suggesting that cardiac fibroblasts are not the source cells of plasma HGF (FIG. 6).

Neutrophils are the most abundant white blood cells in the blood and play a key role in tissue wound healing. According to the literature, serum amyloid A (SAA) can activate neutrophils and induce degranulation. Since the turquoise module is associated with neutrophils and neutrophils infiltrating the myocardial tissue following myocardial infarction, this study explored whether HGF was derived from neutrophils. In this study, neutrophils were isolated from the peripheral blood of mice and stimulated with SAA. Isolation of neutrophils from peripheral blood of C57BL/6J mice (for isolation methods, refer to “Ubags NDJ, Suratt BT. Methods Mol Biol. 2018:1809:45-57.”), neutrophils were inoculated with 6×10⁴ to 2 ml DMEM/F-12 serum-free medium supplemented with 10% (v/v) fetal bovine serum, and 10 μg/mL of SAA was added to the medium to stimulate neutrophils and after addition, incubated at a sterile cell incubator with 37° C., 5% (v/v) CO₂ in for 5 hours. After incubation, the culture supernatant was obtained, and ELISA detected the level of HGF secreted by neutrophils. Measurements showed that HGF in neutrophil culture supernatant increased significantly after stimulation (FIG. 7). These results confirmed that elevated after myocardial infarction was derived from neutrophils.

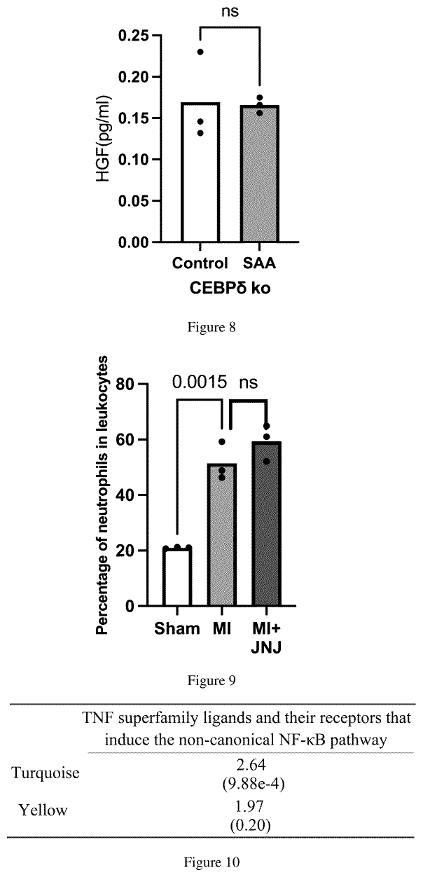

CEBP family transcription factors regulate the production of neutrophil inflammatory cytokines. In the CEBP family, CEBPδ is expressed in mature neutrophils. This study isolated neutrophils from the peripheral blood of Cebpδ knockout mice (purchased from Saiye Corporation, article No. KOCMP-12609-Cebpd-B6J-VA) and stimulated them to replicate the above process. Measurements indicated an almost 500-fold decrease in HGF levels with no change in HGF release following SAA treatment. (FIG. 8). These data suggested that Cebpδ regulates the production of HGF by neutrophils.

It has been reported that the HGF-MET signal is involved in recruiting anti-tumor neutrophils. In this study, the HFG-MET signal was blocked, and neutrophil infiltration was detected in myocardial tissue after myocardial infarction. Six mice with myocardial infarction obtained by LCA were divided into a control group (3 mice) and a treatment group (3 mice). After the end of the group, mice in the treatment group were given MET inhibitor JNJ-38877605 (purchased from MedChemExpress, article No. HY-50683, with 0.1 mL PBS buffer as solvent) by oral gavage at a dose of 50 mg/kg mice weight. Mice in the control group were given the same amount of PBS solvent by oral gavage as the control group. At the end of the gavage, White blood cells (CD45+) and neutrophils (CD45+CD11b+Ly6G+) were isolated from the myocardial tissue on day 1 after myocardial infarction (i.e., day 1 after myocardial infarction surgery) by fluorescence-activated cell sorting (FACS) using BD FACSARIA III flow cytometry. The proportion of neutrophils to white blood cells was calculated. The measurements showed no changes in the total number of neutrophils or the ratio of neutrophils to white blood cells. These data suggested that the HFG-MET signal did not affect the migration and infiltration of neutrophils into myocardial tissue after myocardial infarction (FIG. 9).

These data confirmed that the process of neutrophil release of HGF was essential for cardiac repair after myocardial infarction.

### 3. Neutrophil-Mediated Membrane TNFα Signal Pathway Activates Non-Canonical NF-κB After Acute Myocardial Infarction, Thereby Inducing the Expression of HGF Receptor Met Protein in Fibroblasts

MET receptors were categorized in the turquoise module, indicating that their expression is also linked to HGF signaling or interactions between neutrophils and fibroblasts. Membrane TNFα signals induced non-canonical NF-κB pathways and promoted cell proliferation. The TNF superfamily ligands and their receptors that induce non-canonical NF-KB signal are highly enriched in the turquoise module but not in the yellow module (FIG. 10). Consistent with this observation, proteins interacting with TNFR2 were associated with turquoise modules and proliferating fibroblasts (FIG. 11). These analyses suggested that membrane-bound TNFα expressed by neutrophils binds to TNFR2 in fibroblasts, inducing a non-canonical NF-κB signal and promoting MET expression in fibroblasts. This laid the foundation for the HGF-MET signal.

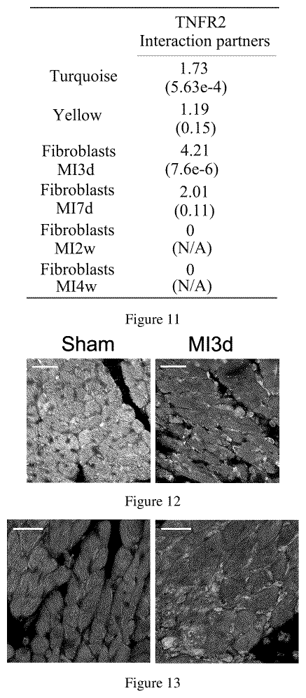

In this study, for staining analysis, myocardial tissue was taken from the mouse model of myocardial infarction on day 3 after acute myocardial infarction (day 3 after myocardial infarction surgery). The results showed that the co-localization of TNF-α and TNFR2 increased significantly on day 3 after myocardial infarction (FIG. 12). Membrane TNFα induced the processing of NF-κB2 precursor protein p100 to p52, and NF-κB2/p52 and RELB mediated non-canonical NF-κB signal. There was a significant increase of NF-κB2 in fibroblasts on day 3 after myocardial infarction (FIG. 13).

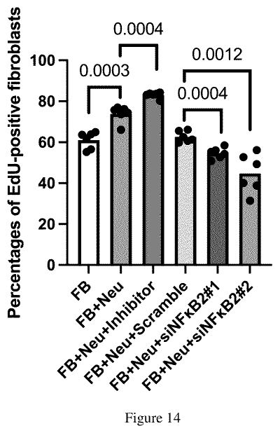

In this study, the non-canonical NF-κB pathway induced by membrane TNF-α was detected in the co-culture system of fibroblasts and neutrophils. Mice model of acute myocardial infarction was prepared, and cardiac fibroblasts and neutrophils were isolated from peripheral blood of C57BL/6J mice on day 3 after acute myocardial infarction (i.e., day 3 after myocardial infarction surgery). Cardiac fibroblasts and neutrophils were inoculated to DMEM/F-12 serum-free medium supplemented with 10% (v/v) fetal bovine serum to co-culture to observe the interaction between fibroblasts and neutrophils. It was found that co-culturing fibroblasts with neutrophils significantly increased the number of proliferating fibroblasts compared to fibroblasts alone (FIG. 14). This proliferative effect may partly be attributed to membrane TNFα signal and neutrophil release of growth factors such as HGF. If the membrane TNFα signal does play a role, then enhancing this signal should increase the proliferation of fibroblasts. Tumor necrosis factor-α converting enzyme (TACE, also known as ADAM-17) was a membrane-bound metalloproteinase that cleaved membrane TNFα and released soluble TNFα. Enhancing membrane TNFα signaling through TACE inhibition significantly increased the proliferating fibroblasts'proportion. (FIG. 14).

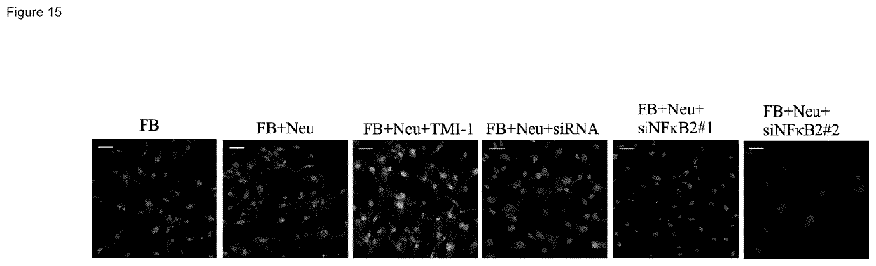

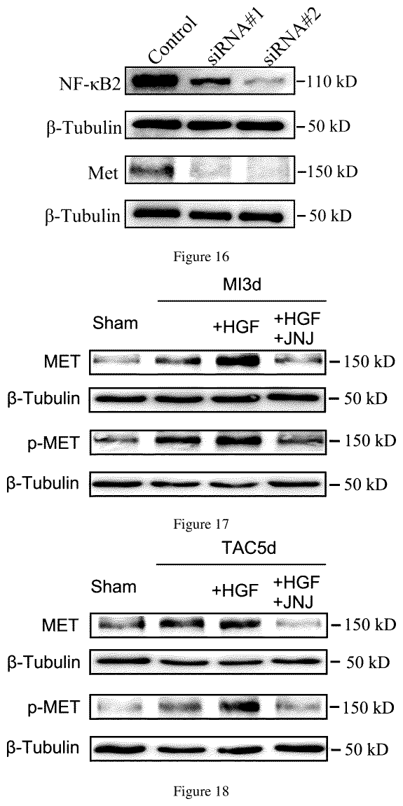

This study further investigated whether NF-κB2/p52 is crucial for fibroblast proliferation. Cardiac fibroblasts were isolated from mice on day 3 after acute myocardial infarction, i.e., day 3 after myocardial infarction surgery, and NF-κB2 knockdown siRNA was delivered to fibroblasts (for the siRNA sequence, refer to “Schumm K et al. EMBO J. 2006 Oct. 18; 25(20):4820-32.”). NF-κB2 knockdown fibroblasts were cultured in DMEM/F-12 serum-free medium supplemented with 10% (v/v) fetal bovine serum, either alone or co-cultured with neutrophils, to observe the proliferation of fibroblasts and their expression of MET. Since primary fibroblasts isolated from mice hearts were difficult to transfection with Lipofectamine, in this study, siRNA knockdown NF-κB2 was delivered to fibroblasts by electroporation (electroporation fibroblasts were suspended in OptiMEM-GlutaMAX solution with a cell density of 1×10⁵, Gene Pulser MXcell electroporation system (Bio-Rad) is powered by 300V, 500 μF, 1000 ohm pulses, exponential attenuation program). This study obtained over 60% electroporation efficiency, and NF-κB2 was significantly knocked down in fibroblasts (FIG. 15). Knocking down NF-κB2 significantly reduced the percentage of proliferating fibroblasts (FIG. 14). Knocking down NF-κB2 significantly reduced MET receptor expression in fibroblasts (FIG. 16).

These data confirmed that membrane TNFα signals induced the non-canonical NF-κB pathway, which mediates fibroblast proliferation.

### 4. HGF Promotes the Proliferation of Cardiac Fibroblasts After Acute Myocardial Infarction

HGF-MET plays a role in developing various tumors and contributes to liver regeneration. HGF binds to MET receptors, induces MET dimerization and autophosphorylation, and activates MET by phosphorylating the activation loop's tyrosine residues at Y1234 and Y1235. Phosphorylation of Y1234 and Y2235 can therefore be used as a marker of MET activation. To confirm whether HGF can promote the proliferation of cardiac fibroblasts after acute myocardial infarction, six mice models of myocardial infarction obtained by permanent LCA were divided into the control group (6 mice) and treatment group (6 mice). After grouping, mice in the treatment group were given MET inhibitor JNJ-38877605 (purchased from MedChemExpress, article No. HY-50683, with 0.1 mL PBS buffer as solvent) by oral gavage at a dose of 50 mg/kg mice weight. Mice in the control group were given the same amount of PBS solvent by oral gavage. Oral gavage administration was performed once a day from the day of operation until 3 days after myocardial infarction surgery. After oral gavage administration, an antibody against MET and phosphorylated MET (Y1234/Y1235) was used to perform western blot analysis on fibroblasts on day 3 after myocardial infarction surgery to detect MET levels and phosphorylated MET(Y1234/Y1235) levels. MET and phosphorylated MET(Y1234/Y1235) were found to be increased on day 3 after myocardial infarction. MET inhibitors effectively block the HGF-MET signal (FIG. 17). Similar findings were found when the above experimental process was repeated in the TAC model (FIG. 18). These data suggested that HGF activated MET in fibroblasts in both myocardial infarction (MI) models of LCA and TAC models.

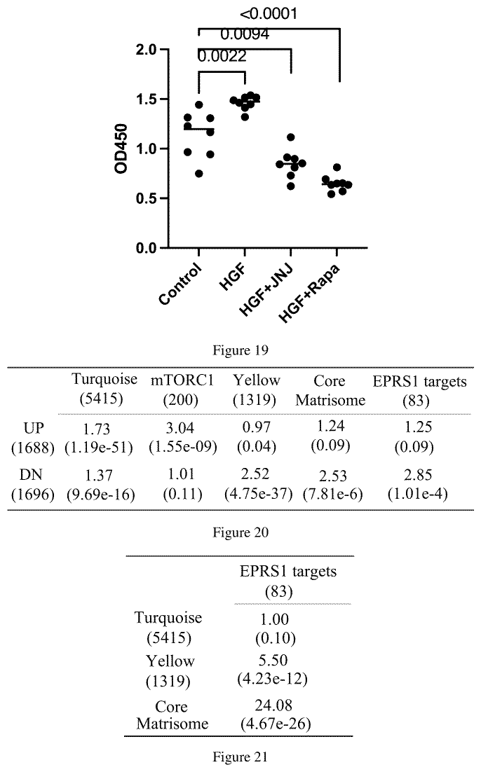

Subsequently, this study examined whether HGF directly promoted the proliferation of cardiac fibroblasts. Six mice models of myocardial infarction were obtained by LCA, and cardiac fibroblasts were isolated from the mice on day 3 after acute myocardial infarction (i.e., day 3 after myocardial infarction surgery) (for isolation methods, refer to “Pramod Sahadevan, Bruce G Allen, Methods. 2022 July; 203:187-195.”), the fibroblasts were inoculated with 2×10⁵ cells into 2 ml DMEM/F-12 serum-free medium supplemented with 10% (v/v) fetal bovine serum and then cultured in a sterile cell incubator at 37° C. and 5% (v/v) CO₂ for 48 hours. After the culture, the fibroblasts were transferred to serum-free DMEM/F-12 serum-free medium and cultured in a sterile cell incubator at 37° C. and 5% (v/v) CO₂ for 24 hours. After the culture was completed, 60 ng/mL of HGF, 0.1 μM of MET inhibitor JNJ-38877605, and 0.05 μM of rapamycin were added to the culture system to treat cardiac fibroblasts, which were then incubated in a sterile cell incubator at 37° C. and 5% (v/v) CO₂ for 72 hours after the addition. After incubation, the number of living cells was measured using a sensitive colorimetric assay (Cell Counting Kit 8, AC11L054, LFF23063, Life-iLab) to compare cell proliferation. It was found that HGF significantly promoted fibroblast proliferation, while inhibition of MET or mTORC1 signal completely blocked HGF's role in promoting fibroblast proliferation (FIG. 19).

These data showed that the HFF-MET signal promoted the proliferation of cardiac fibroblasts after myocardial infarction.

### 5. RNA Sequencing Suggested that HGF may Promote Fibroblast Proliferation by Activating the AKT-mTORC1 Pathway but Inhibit Collagen Synthesis In Myofibroblasts

To find clues on how HGF regulates fibrosis, cardiac fibroblasts treated with 60 ng/mL HGF in the above experiment were taken. mRNA was extracted, and RNA-seq was performed by the Illumina sequencing platform. In this study, 1688 genes were up-regulated, and 1696 were down-regulated. The up-regulated gene is more enriched in the turquoise module than the down-regulated gene, suggesting that HGF promotes the turquoise module (FIG. 20). AKT-mTORC1 is an essential downstream pathway that promotes cell proliferation. Genes related to the mTORC1 pathway are up-regulated in gene enrichment, indicating that HGF directly activates AKT-mTORC1 in fibroblasts (FIG. 20). On the contrary, the down-regulated gene was highly enriched in the yellow module, suggesting that HGF inhibited the function of the yellow module. Core extracellular matrix-related genes are highly enriched in down-regulated genes (FIG. 20).

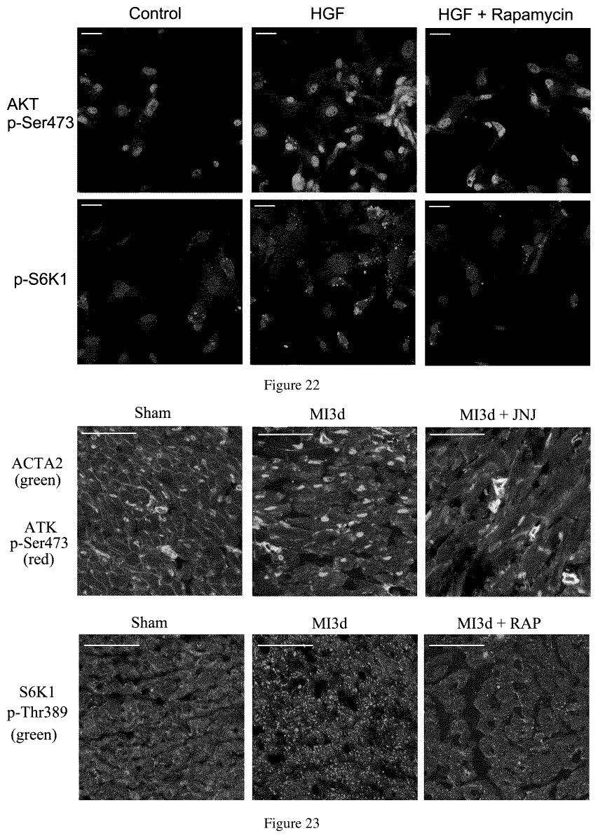

ECM proteins, especially collagen, are rich in proline. Proline and hydroxyproline constitute about 25% of the amino acids in collagen. EPRS1 (glutamyl-prolyl tRNA synthetase) controls the translation of these ECM proteins. EPRS1 catalyzes the proline (Pro) attachment to the corresponding tRNA to synthesize Pro-containing proteins, which play a key role in cardiac fibrosis by promoting the synthesis of Pro-rich fibrotic proteins. Eighty-three genes containing Pro-Pro motifs were identified as preferred regulatory targets for EPRS1. The regulatory targets of EPRS1 were enriched in genes down-regulated after HGF treatment. These regulatory targets of EPRS1 are highly enriched in the core extracellular matrix and yellow module (FIG. 21).

These data and analyses suggested that HGF activated the AKT-mTORCI pathway to promote fibroblast proliferation but inhibited myofibroblast function via EPRS1.

### 6. HGF Activates the AKT-mTORC1 Pathway to Promote Fibroblast Proliferation

Growth factors were bound to extracellular regions of their receptor tyrosine kinases (RTKs) to activate RTKs. The main downstream pathway of RTKs is the PI3K-AKT-mTORC1 pathway. Phosphorylation of AKT Ser473 marks the activation of this kinase. p70 S6 kinase is a substrate for mTORC1, and phosphorylated p70 S6 kinase (Thr389) can be used as a marker for mTORC1 activation. To verify the mechanism of HGF regulation of fibrosis, cardiac fibroblasts treated with 60 ng/mL of HGF and 0.05 μM of rapamycin in the above experiment were immunofluorescent stained with antibodies against activated forms of AKT and p70 S6 kinases. Phosphorylation at Thr389 of p70 S6 kinase and at Ser473 of AKT was used to assess mTORC1 and AKT activation. The results showed that these two kinases were activated in fibroblasts after HGF treatment. Rapamycin is an inhibitor of mTORC1 and inhibits HGF-induced phosphorylation of p70 S6 kinase (FIG. 22).

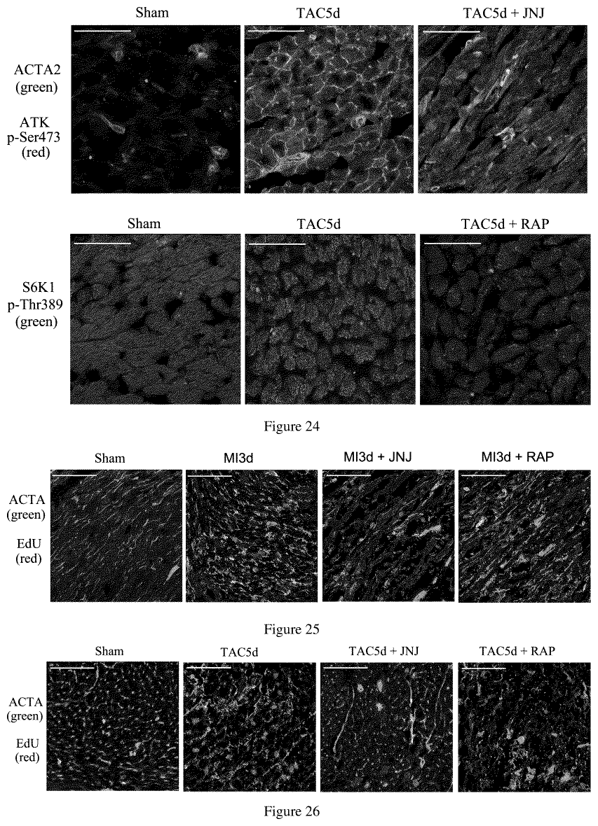

This study further verified the HGF-induced AKT-mTORC1 pathway in MI models of LCA and TAC models. IHC analysis was performed on left ventricular tissues on day 3 after myocardial infarction (i.e., day 3 after myocardial infarction surgery) and on day 5 after TAC. We found that the MET inhibitor JNJ inhibits the HGF signal and completely blocks AKT and mTORC1 activation. Rapamycin also blocks the phosphorylation of p70 S6 kinase (FIG. 23, FIG. 24). This study further investigated the role of the HFG-MET-Akt-MTORC1 pathway in fibroblast proliferation. In this study, a novel thymidine analog EdU was used to label proliferating cells in myocardial tissue (For labeling methods, refer to Fu X et al. J Clin Invest. 2018 May 1; 128(5):2127-2143”). MET inhibitors or rapamycin treatment significantly inhibited fibroblast proliferation on day 3 after myocardial infarction (FIG. 25). MET inhibitors or rapamycin treatment also considerably inhibited TAC model cardiac fibroblast proliferation (FIG. 26).

### 7. HGF Inhibits Collagen Synthesis in Myofibroblasts via mTORC1-S6K1-Mediated Phosphorylation of EPRS1

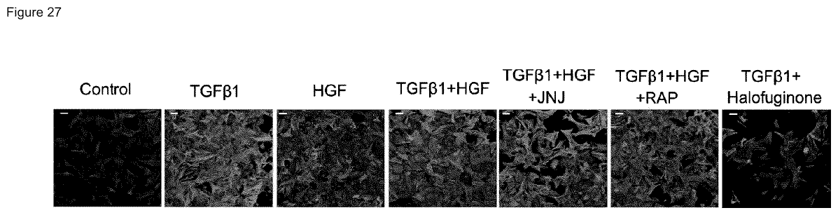

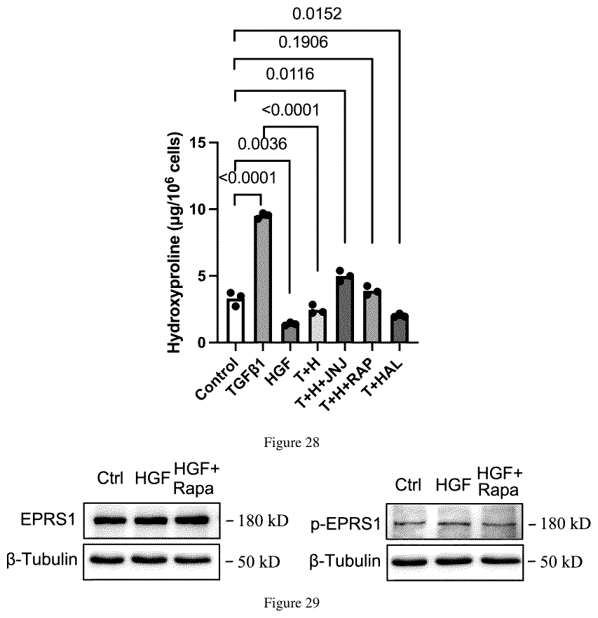

TGFβ1 promotes the differentiation of fibroblasts into contractile myofibroblasts. To determine the role of HGF in regulating myofibroblasts, TGFβ1 was used to induce fibroblasts to differentiate into myofibroblasts. Six mice models of myocardial infarction were obtained by LCA, and cardiac fibroblasts were isolated from the mice on day 3 after acute myocardial infarction (i.e., day 3 after myocardial infarction surgery) (for isolation methods, refer to “Pramod Sahadevan, Bruce GAllen, Methods. 2022 July; 203:187-195.”). The fibroblasts were inoculated with 2×10⁵ cells into 2 ml DMEM/F-12 serum-free medium supplemented with 10% (v/v) fetal bovine serum and cultured in a sterile cell incubator at 37° C. and 5% (v/v) CO₂ for 48 hours. The fibroblasts were transferred to serum-free DMEM/F-12 serum-free medium and cultured in a sterile cell incubator at 37° C. and 5% (v/v) CO₂ for 24 hours. After the culture was completed, 600 pmol/L TGFβ1 (purchased from MedChemExpress, HY-P70648), 60 ng/mL of HGF, 0.1 μM of MET inhibitor JNJ-38877605, 0.05 μM of RAP (rapamycin, mTORC1 inhibitor) and 50 ng/mL Halofuginone (EPRS1 inhibitor, purchased from Bide Pharmatech Ltd., article No. BD132622) were added to cardiac fibroblasts at 37° C., 5% (v/v) CO₂. After incubation, the antibody against ACTA2 was used for immunofluorescence staining of the fibroblasts. The incubation media was taken, and a hydroxyproline analysis kit (BC0250, Solarbio) was used to determine the hydroxyproline content. The results showed that HGF treatment significantly inhibited the function of myofibroblasts, and MET inhibitors or rapamycin blocked the action of HGF (FIG. 27). Collagen consists of either Gly-Pro-X (X is any amino acid) or Gly-X-Hyp (hydroxyproline) repeats, with hydroxyproline accounting for about 14% of collagen's dry weight. Due to its abundance, measuring hydroxyproline can provide a quantitative comparison of collagen content. Collagen synthesis was significantly inhibited by HGF treatment (FIG. 28).

Six mice models of myocardial infarction were obtained by LCA, and cardiac fibroblasts were isolated from the mice on day 7 after acute myocardial infarction (i.e., on day 7 after myocardial infarction surgery) (for isolation methods, refer to “Pramod Sahadevan, Bruce G Allen, Methods. 2022 July; 203:187-195.”). Fibroblasts were inoculated with 2×10⁵ cells into 2 ml of serum-free medium DMEM/F-12 supplemented with 10% (v/v) fetal bovine serum and cultured in a sterile cell incubator with 5% (v/v) CO₂ at 37° C. for 4 hours. After the culturing, cardiac fibroblasts were treated with 60 ng/mL of HGF, 0.1 μM of MET inhibitor JNJ-38877605, and 0.05 μM of rapamycin, respectively. Cardiac fibroblasts were incubated in a sterile cell incubator at 37° C., 5% (v/v) CO₂ for 72 hours. After incubation, western blotting was performed with antibodies against EPRS1 and its phosphorylated form. Data analysis showed that HGF treatment inhibited downstream regulatory targets of EPRS1. Recent studies have shown that EPRS1 is a direct substrate for p70 S6 kinase. EPRS1 encodes a bifunctional glutamyl-prolyl tRNA synthetase. Upon activation by p70 S6 kinase, phosphorylation of Ser999 of EPRS1 induces its dissociation from the aminoacyl tRNA synthetase multienzyme complex, thereby reducing the translation of proline-rich mRNA. Activation of the HGF-MET pathway enhances phosphorylation of EPRS1 (Ser999), an effect that rapamycin can block (FIG. 29).

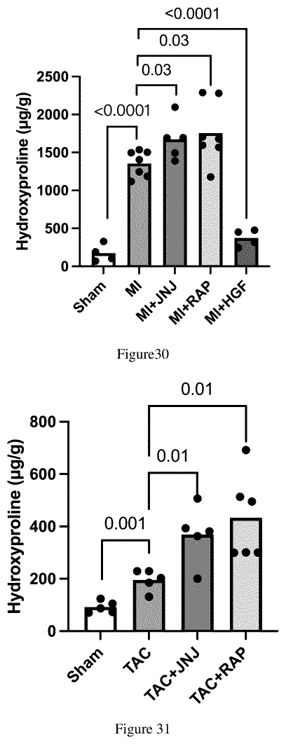

This study further verified whether the HGF signal inhibits the function of myofibroblasts in vivo. Nineteen mice models of myocardial infarction obtained by LCA were divided into three groups. The three groups were the control group (7 mice), the MET inhibitor intervention group (5 mice), and the rapamycin intervention group (7 mice). After grouping, mice in the MET inhibitor intervention group were given MET inhibitor JNJ-38877605 by oral gavage at a dose of 50 mg/kg mice weight (MET inhibitor purchased from MedChemExpress, article No. HY-50683, with 0.1 mL PBS buffer as solvent). Mice in the rapamycin intervention group were intraperitoneally injected with rapamycin at a dose of 10 mg/kg mice weight (rapamycin was purchased from MedChemExpress, article No. HY-10219, with 0.1 mL PBS buffer as solvent). Mice in the control group were given the same amount of PBS solvent, and the intervention was performed once a day from day 3 to day 10 after myocardial infarction surgery. After the intervention, a hydroxyproline analysis kit (BC0250, Solarbio) was used to detect the hydroxyproline content in the left ventricle of mice on the 10th day after myocardial infarction (i.e., the 10th day after myocardial infarction surgery). The results showed that blocking the HGF signal pathway with MET inhibitors or rapamycin significantly increased hydroxyproline content in an acute myocardial infarction model (FIG. 30). The above experimental process was repeated in the TAC model, during which the MET inhibitor and rapamycin were treated from day 7 to day 15 after TAC, and it was found that inhibition of the HGF signal pathway also resulted in a significant increase in hydroxyproline content (FIG. 31).

### 8. Neutrophils Release HGF to Control Myocardial Fibrosis and Myocardial Injury Repair after Acute Myocardial Infarction

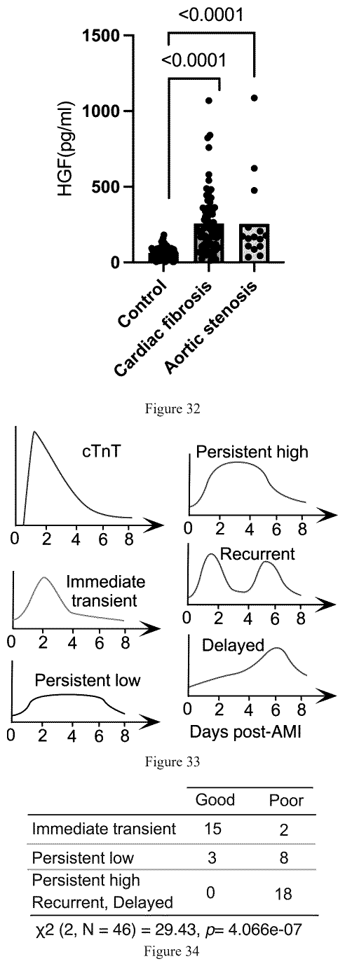

The diagnosis of cardiac fibrosis largely relies on invasive tissue biopsy or detection by cardiovascular magnetic resonance late gadolinium enhanced (LGE) and T1 mapping. Because fibrosis involves many cell types, the discovery of plasma markers of cardiac fibrosis is of great clinical value. So far, several molecular markers of fibrosis have been reported in the literature. Still, it is thought that these markers neither originate from the heart nor are known to reflect specific stages of fibrosis. HGF is associated with the turquoise module that is linked to proliferating cardiac fibroblasts. In this study, patients (100 patients from FUWAI CENTRAL CHINA CARDIOVASCULAR HOSPITAL) diagnosed clinically with myocardial fibrosis and assessed through cardiac magnetic resonance gadolinium delayed enhancement were selected. HGF was significantly elevated in the plasma of patients with cardiac fibrosis (FIG. 32). In patients with aortic stenosis, cardiac afterload increases, leading to cardiac remodeling and myocardial fibrosis. This study selected patients (15 patients from Fuwai Central China Cardiovascular Hospital) with aortic stenosis, and plasma HGF levels were also significantly elevated in patients with aortic stenosis before surgery (FIG. 32).

After acute myocardial infarction, cardiac injury repair is essential to restore cardiac function. Within 2 days after an acute myocardial infarction, necrotic cardiac muscle cells release a large amount of troponin, and a sharply elevated level of troponin T or troponin I in the blood is one of the criteria for clinical diagnosis of acute myocardial infarction. The study monitored the dynamic variations of HGF levels in 46 patients with acute myocardial infarction (ST elevation and non-ST elevation) collected from FUWAI CENTRAL CHINA CARDIOVASCULAR HOSPITAL. In this study, we found that the dynamic variations of HGF can be roughly divided into five categories: immediate transient elevation (significant elevation within 3 days after myocardial infarction, and then decrease), persistent low-level elevation, persistent high-level elevation (more than three consecutive days, 10 folds higher than the normal population), recurrent and delayed elevation (FIG. 33). The results of this study have also confirmed that an immediate transient increase of plasma HGF represented a normal inflammatory response (FIG. 5). Persistent low-level elevation and other types should be considered abnormal inflammatory responses.

To investigate whether dynamic variations of HGF can predict the prognosis of hospitalized patients with acute myocardial infarction, the study divided dynamic variations of HGF into three groups: immediate transient elevation, persistent low-level elevation, and other types of groups (persistent high-level elevation, recurrence, and delayed elevation). To evaluate the prognosis of patients with acute myocardial infarction during hospitalization, the following four conditions were considered as heart failure events after acute myocardial infarction: 1) new heart failure; 2) deterioration of heart failure (progression of Killip scale from II to III/IV, or from III to IV); 3) cardiogenic shock; 4) death from cardiogenic shock or heart failure. The prognosis was good when the patient presented with one of the following two conditions: 1) left ventricular ejection fraction (EF)>50% at discharge and no heart failure events occurred; 2) the patient's EF at discharge was between 40% and 50%, but improved during hospitalization, and there were no heart failure events during hospitalization. Patients with any of the following conditions were judged to have a poor prognosis: 1) EF was lower than 40% at discharge; 2) The EF value of the patient was between 40% and 50% at discharge but decreased during hospitalization; 3) there were heart failure events during hospitalization. This study analyzed the correlation between the dynamic variations of HGF after acute myocardial infarction and the prognosis of patients with acute myocardial infarction during hospitalization. The analysis found that among the monitored 46 patients with acute myocardial infarction, the group with immediate transient elevation was significantly associated with a good prognosis. In contrast, the other groups were significantly associated with a poor prognosis (FIG. 34).

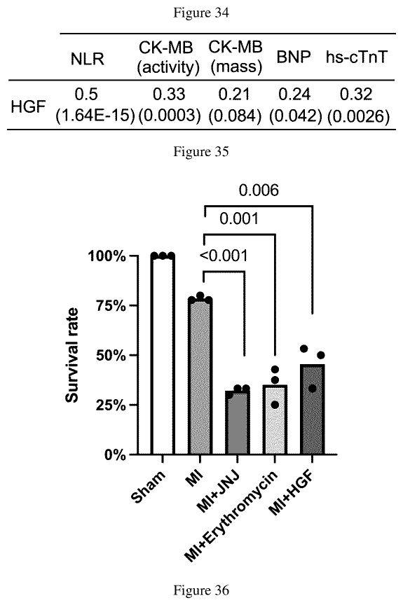

Neutrophil to lymphocyte ratio (NLR) can reflect the inflammatory state of the body to a certain extent, brain natriuretic peptide (BNP) is a recognized marker of heart failure, creatine kinase myocardial type (CK-MB) mass and activity, hypersensitive cardiac troponin T (hs-cTnT) reflect cardiac muscle cell injury. These clinical indicators can reflect the status of patients with myocardial infarction to a certain extent. This study analyzed the correlation between HGF and clinical biochemical indicators (NLR, BNP, CK-MB mass and activity, and hs-cTnT) detected after AMI. It was found that HGF was moderately correlated with NLR (Spearman correlation coefficient 0.5). This further confirmed that the HGF level can accurately reflect the activation state of neutrophils to reflect the inflammatory repair of patients with myocardial infarction. However, the correlation between HGF and cardiac muscle cell markers was low (FIG. 35).

To investigate whether the HGF signal pathway is crucial in repairing myocardial injury after acute myocardial infarction, MET inhibitors were injected into a mouse model of myocardial infarction to block the HGF signal. Fifty-three mice models of myocardial infarction obtained by LCA were divided into six groups. The six groups were divided into three control groups (28 mice divided into three groups) and three treatment groups (25 mice divided into three groups). After grouping, mice in treatment group were given MET inhibitor JNJ-38877605 by oral gavage at a dose of 50 mg/kg mice weight (MET inhibitor purchased from MedChemExpress (HY-50683), with 0.1 mL PBS buffer as solvent), the control group was given the same amount of PBS solvent by oral gavage, starting from day 1 after myocardial infarction surgery, the mice were orally administered once a day for seven consecutive days, and the survival details were observed. Studies have confirmed that a moderate HGF signal is critical for cardiac repair after myocardial infarction. In the acute myocardial infarction mice model, the survival rate of mice in the control group was 78%. In comparison, the survival rate of mice with myocardial infarction in the treatment group was about 32%. Thus, blocking the HGF signal pathway with MET inhibitors significantly reduced the survival rate of mice (FIG. 36).

This study found that erythromycin can inhibit neutrophil tissue infiltration and thus inhibit the inflammatory response of injured tissues. Still, erythromycin does not significantly affect neutrophil degranulation (FIG. 5). To further investigate whether inhibiting neutrophil infiltration affects the survival of myocardial infarction mice, the myocardial infarction mice model was injected with erythromycin. Fifty-six mice models of myocardial infarction obtained by LCA were divided into six groups: three control groups and three treatment groups. After grouping, the mice in the three treatment groups were injected with erythromycin at a dose of 100 mg/kg mice weight (purchased from MedChemExpress, article No. HY-B0220, with 0.1 mL PBS buffer as solvent), the control group was injected with the same amount of PBS solvent intraperitoneally, and the intervention was performed once a day from day 1 to day 6 after myocardial infarction surgery. After the intervention, the survival details of the mice were observed. It was found that the administration of erythromycin significantly reduced survival in mice, further supporting the idea that inhibiting inflammation is detrimental to cardiac repair. (FIG. 36).

To explore how excessive HGF signal pathway affects repair after acute myocardial infarction, HGF was injected from day 4 to day 7 after myocardial infarction surgery. HGF strongly inhibited myocardial fibrosis (FIG. 30). To further confirm this result, 53 mice models of myocardial infarction obtained by LCA were divided into three control and three treatment groups. Three treatment group mice were injected with HGF through the tail vein at a dose of 250 ug/kg mice weight (purchased from R&D, article No. 2207-HG-025, with 0.1 mL PBS buffer as solvent). The control group was injected with the same amount of PBS solvent in the tail vein, and the intervention was performed once a day from day 1 to day 3 after myocardial infarction. After the intervention, the survival details of the mice were observed. It was found that HGF injection from day 4 to day 6 after myocardial infarction significantly increased mortality in mice because it inhibited cardiac repair (FIG. 36). These results suggested that persistently elevated HGF was also detrimental to cardiac repair. All myocardial infarction models were examined by anatomic and pathological examination, and the results confirmed that these acute myocardial infarction models died of heart rupture.

The embodiments mentioned above are only examples for precise description and are not intended to limit the implementation manner. For those of ordinary skill in the art, changes or modifications in other forms can also be made based on the above description. There is no need and cannot be exhaustive of all implementations here. The apparent changes or changes derived from this are still within the protection scope of the present application.

1. A molecular marker, wherein the molecular marker comprises hepatocyte growth factor and the molecular marker has any one of the following functions:
(a) diagnosis of cardiac fibrosis;
(b) evaluation of the severity of cardiac fibrosis;
(c) prognosis evaluation of acute myocardial infarction; and
(d) prognosis evaluation of cardiovascular diseases with concomitant cardiac fibrosis other than acute myocardial infarction.

2. The molecular marker, according to claim 1, wherein the cardiac fibrosis comprises cardiac fibrosis caused by pressure load and/or cardiac fibrosis caused by myocardial infarction and
cardiovascular diseases with concomitant cardiac fibrosis other than acute myocardial infarction comprise hypertension, diastolic heart failure, cardiomyopathy, and/or sudden cardiac death.

3. Use of a reagent for detecting the molecular marker of claim 1 in a sample to be tested in the preparation of a product, wherein the product has any one of the following functions:
(a) diagnosis of cardiac fibrosis;
(b) evaluation of the severity of cardiac fibrosis;
(c) prognosis evaluation of acute myocardial infarction; and
(d) prognosis evaluation of cardiovascular diseases with concomitant cardiac fibrosis other than acute myocardial infarction.

4. A product, wherein the product comprises a reagent for detecting the molecular marker of claim 1 in a sample to be tested; the product has any one of the following functions:
(a) diagnosis of cardiac fibrosis;
(b) evaluation of the severity of cardiac fibrosis;
(c) prognosis evaluation of acute myocardial infarction; and
(d) prognosis evaluation of cardiovascular diseases with concomitant cardiac fibrosis other than acute myocardial infarction.

5. A prediction model wherein the prediction model has any one of the following functions:
(a) diagnosis of cardiac fibrosis;
(b) evaluation of the severity of cardiac fibrosis;
(c) prognosis evaluation of acute myocardial infarction; and
(d) prognosis evaluation of cardiovascular diseases with concomitant cardiac fibrosis other than acute myocardial infarction;
wherein the prediction model comprises a detection module, a data analysis module, and a prediction module;
wherein the detection module is used for detecting the level of the molecular marker of claim 1 in a sample to be tested;
the data analysis module is used for analyzing the detection results output by the detection module and
the prediction module classifies whether the sample to be tested is diseased or has a good or poor prognosis based on the analysis results output by the data analysis module.

6. A method for screening drug, wherein the drug comprises a drug for treating cardiac fibrosis, acute myocardial infarction, or cardiovascular diseases with concomitant cardiac fibrosis other than acute myocardial infarction; and
the method includes administering the drug to be screened to the experimental subject, obtaining the results related to the molecular markers of claim 1 after administering the drug to the experimental subjects, and determining whether the drug to be screened is effective or ineffective based on the obtained results.

7. A method for preventing or treating cardiac fibrosis, comprising administering any one or a combination of hepatocyte growth factor, hepatocyte growth factor signal pathway blocker, and neutrophil infiltration inhibitor to a subject in need.

8. The method, according to claim 7, wherein the cardiac fibrosis comprises cardiac fibrosis caused by pressure load, cardiac fibrosis induced by myocardial infarction, or cardiac fibrosis concomitant with cardiovascular diseases other than acute myocardial infarction.

9. The method, according to claim 8, wherein cardiovascular diseases other than acute myocardial infarction comprise hypertension, diastolic heart failure, cardiomyopathy, and/or sudden cardiac death.

10. The method, according to claim 8, wherein when the cardiac fibrosis is caused by myocardial infarction, the method comprises administering a hepatocyte growth factor to a subject experiencing acute myocardial infarction or administering a hepatocyte growth factor signal pathway blocker and/or a neutrophil infiltration inhibitor to a subject experiencing acute myocardial infarction.

11. The method, according to claim 8, wherein when the cardiac fibrosis is concomitant with cardiovascular diseases other than acute myocardial infarction, the method comprises administering a hepatocyte growth factor and/or hepatocyte growth factor signal pathway blocker to the subject experiencing cardiac fibrosis.

12. The method according to claim 8, wherein the method comprises:
if the plasma hepatocyte growth factor level in a subject experiencing acute myocardial infarction after treatment is persistent high-level elevation or recurrence, administering hepatocyte growth factor signal pathway blocker and/or neutrophil infiltration inhibitor to the subject,
if the plasma hepatocyte growth factor level in a subject experiencing acute myocardial infarction after treatment is persistent low-level elevation, administering a hepatocyte growth factor to the subject and
if the plasma hepatocyte growth factor level in a subject experiencing acute myocardial infarction after treatment is delayed elevation, administering a hepatocyte growth factor signal pathway blocker to the subject to adjust the activation time of the hepatocyte growth factor signal pathway to improve the subject's prognosis.

13. The method according to claim 10, wherein when an acute myocardial infarction occurs, the method comprises administering hepatocyte growth factor to the subject experiencing the acute myocardial infarction, or administering a hepatocyte growth factor signal pathway blocker and/or neutrophil infiltration inhibitor to the subject experiencing the acute myocardial infarction to control fibroblast proliferation dominated by the acute inflammatory response and/or control myofibroblast-mediated fibrosis repair.

14. The method according to claim 7, wherein the hepatocyte growth factor signal pathway blocker comprises one or a combination of a hepatocyte growth factor inhibitor, an AKT inhibitor, a mTORC1 inhibitor, and a MET inhibitor.

15. The method according to claim 7, wherein the dose of hepatocyte growth factor is 0.6 to 2.4 mg/m²/day, administered intravenously continuously for 12 to 14 days, wherein m² refers to the surface area of the human body,
the dose of the hepatocyte growth factor signal pathway blocker or neutrophil infiltration inhibitor is 10 mg to 4 g per day, or 60 mg to 4 g per day, or 1 g to 4 g per day, or 10 to 60 mg per day, or 60 mg to 1 g per day.

16. The method according to claim 7, wherein the hepatocyte growth factor signal pathway blocker is the MET inhibitor JNJ-38877605.

17. A drug wherein the drug has any one of the following functions:
(a) prevention and/or treatment of cardiac fibrosis;
(b) prevention and/or treatment of acute myocardial infarction and
(c) prevention and/or treatment of cardiovascular diseases with concomitant cardiac fibrosis other than acute myocardial infarction and
the components of the drug comprise hepatocyte growth factor, hepatocyte growth factor signal pathway blocker, and/or neutrophil infiltration inhibitor.

18. A method for diagnosing and/or evaluating cardiac fibrosis, the method comprises:
determining whether a subject has cardiac fibrosis based on the concentration of hepatocyte growth factor in plasma, if the concentration of hepatocyte growth factor in the subject's plasma is much higher than that of the normal population, it is determined that the subject has cardiac fibrosis; if the concentration of hepatocyte growth factor in the subject's plasma is close to that of the normal population, it is determined that the subject has no cardiac fibrosis.

19. A method for evaluating the prognosis of acute myocardial infarction, the method comprises:
judging the prognosis of a subject based on variations in the concentration of hepatocyte growth factor in plasma, if the concentration of hepatocyte growth factor in the subject's plasma temporarily elevates in an immediate transient manner, the subject's prognosis is good; if the concentration of hepatocyte growth factor in the subject's plasma is persistent high-level elevation, persistent low-level elevation, recurrence, or delayed elevation, the subject's prognosis is poor.

20. A method for improving the prognosis of a subject experiencing acute myocardial infarction, the method comprises:
1) evaluating the prognosis of acute myocardial infarction, comprising judging the prognosis of the subject after treatment based on variations in the concentration of hepatocyte growth factor in plasma, if the concentration of hepatocyte growth factor in the subject's plasma temporarily elevations in an immediate transient manner, the subject's prognosis is good; and if the concentration of hepatocyte growth factor in the subject's plasma persistent high-level elevation, persistent low-level elevation, recurrence or delayed elevation, the subject's prognosis is poor; and
2) administering a hepatocyte growth factor, a hepatocyte growth factor signal pathway blocker, or a neutrophil infiltration inhibitor to the subject with poor prognosis, comprising:
when the hepatocyte growth factor level in the subject after treatment is persistent high-level elevation or recurrence, administering hepatocyte growth factor signal pathway blocker or neutrophil infiltration inhibitor to the subject; when the plasma hepatocyte growth factor level in the subject with acute myocardial infarction after treatment is persistent low-level elevation, administering a hepatocyte growth factor to the subject; and when the hepatocyte growth factor level in the subject after treatment delayed elevation, administering a hepatocyte growth factor signal pathway blocker to the subject to adjust the activation time of the hepatocyte growth factor signal pathway to improve the subject's prognosis.
